(19) United States
(12) Patent Application Publication
(10) Pub. No.: US 2021/0338094 A1
(43) Pub. Date: Nov. 4, 2021

(54) ELECTROCARDIOGRAM DETECTION DEVICE

(71) Applicant: Quanta Computer Inc., Taoyuan City (TW)

(72) Inventor: Chih-Hsiung YU, Taoyuan City (TW)

(21) Appl. No.: 17/026,626

(22) Filed: Sep. 21, 2020

(30) Foreign Application Priority Data
May 4, 2020 (TW) 109114751

(51) Int. Cl. A61B 5/04; A61B 5/0472; A61B 5/00; A61B 5/044

(52) U.S. Cl. CPC A61B 5/04004; A61B 5/044; A61B 5/0006; A61B 5/0472

(57) ABSTRACT

An electrocardiogram detection device is provided. The electrocardiogram detection device includes a filter amplifier and a microcontroller. The filter amplifier filters and amplifies a raw heartbeat signal of a user. The microcontroller is coupled to the filter amplifier. The microcontroller generates a control signal according to a trigger signal to control the filter amplifier to filter out components of the raw heartbeat signal in a specific bandwidth range.

### CROSS REFERENCE TO RELATED APPLICATIONS

This application claims priority of Taiwan Patent Application No. 109114751, filed on May 4, 2020, the entirety of which is/are incorporated by reference herein.

### BACKGROUND OF THE INVENTION

### Field of the Invention

This invention relates to an electrocardiogram detection device, and in particular relates to an electrocardiogram detection device in which a detection frequency can be adjusted according to a user's current usage situation.

### Description of the Related Art

An electrocardiogram (ECG) is an image technology that records the physiological activity of the heart of a patient in units of time, which is captured and recorded through electrodes contacting the skin of the chest cavity of the patient. The working principle of the electrocardiogram is that every time the heart beats, the depolarization of the myocardial cells will cause a small electrical change on the skin surface. This small change is captured and enlarged by the electrocardiogram recording device for depict an electrocardiogram.

When a myocardial cell is at a quiescent state, there is a potential difference formed by a difference between a concentration of positive ions and a concentration of negative ions on both sides of the membrane of the myocardial cell. Depolarization is a process in which the potential difference of the myocardial cell rapidly changes to zero to cause contraction of the myocardial cell.

In a beating cycle of a healthy heart, a depolarized wave generated by a sinoatrial nod cell is transferred through the heart in sequence, first to the entire atrium and then to the ventricle through an “intrinsic conduction path”.

In the prior art, an electrocardiogram can reflect the rhythm of the entire heartbeat, as well as the weak part of the myocardium. Generally, more than two electrodes can be placed on the limbs, and they form a pair of electrodes for detection (for example, a left arm electrode (LA), a right arm electrode (RA), a left leg electrode (LL) can be combined in this way: LA and RA, LA and LL, RA and LL). The output signal of each pair of electrodes is referred to as one lead. Through several leads, the changes in the current of the heart can be observed in different angles. In this process, the tiny change in the voltage between the two electrodes can be recorded and can be displayed on an electrocardiogram paper or monitor for further monitoring of the physiological activity of the heart.

The types of ECG can be distinguished by leads, such as three-lead ECG, five-lead ECG, and twelve-lead ECG, etc. A twelve-lead ECG is the most common clinical type. It can record the potential changes of twelve leads on the body surface at the same time, and depict the signals of the twelve leads on the ECG paper. It is often used for one-time ECG diagnosis.

As for a three-lead ECG and a five-lead ECG, they are mostly used in situations where continuous monitoring of ECG activity by a monitor is required, for example, a situation where a surgery is performed or a situation where monitoring is needed during an ambulance is transporting a patient. Thus, depending on the instruments and the usage situations, there may be different lead-monitoring manners.

An electrocardiogram is the best method for measuring and diagnosing abnormal heart rhythm. It is used to diagnose abnormal heart rhythm when the electrocardiographic conduction tissue is damaged and to diagnose changes in heart rhythm due to electrolyte imbalance.

In different monitoring situations, an ECG detection can be implemented using different forms of electrodes. For example, the current ECG detection manners are roughly divided into the following two types: a conductive gel manner and a metal electrode manner, each of which has its own suitable use situations.

The manner where a patch is used to contact a human body through the conductive gel is suitable for a long-term detection situation, such as the heartbeat monitoring of the elderly. The advantage of the conductive gel is that the contact impedance between the conductive gel and the human body is small. As for the metal electrode, it is suitable for use when the user is temporarily uncomfortable or when the ECG is detected only at a fixed time. The advantage of the metal electrode is that the metal electrode is easier to clean.

### BRIEF SUMMARY OF THE INVENTION

The present invention provides an electrocardiogram detection device which can adaptively adjusts a frequency range of a heartbeat signal detected by the electrocardiogram detection device according to the user's current usage situation to reduce the time waiting for the display of the electrocardiogram, which allows the user to more efficiently obtain an electrocardiogram that reflects his own heartbeat signal.

An embodiment of the present invention provides an electrocardiogram detection device, which at least comprises a filter amplifier and a microcontroller. The filter amplifier filters and amplifies a raw heartbeat signal of a user. The microcontroller is coupled to the filter amplifier. The microcontroller generates a control signal according to a trigger signal to control the filter amplifier to filter out components of the raw heartbeat signal in a specific bandwidth range.

In an embodiment of the invention, the electrocardiogram detection device further comprises a signal collection unit. The signal collection unit is connected to the filter amplifier. The signal collection unit collects the raw heartbeat signal and transmits the raw heartbeat signal to the filter amplifier. Different values of the trigger signal correspond to different types of the signal collection unit.

In an embodiment of the present invention, in response to the signal collecting unit being a gel electrode, the specific frequency range is from 0.05 Hz to 40 HZ.

In an embodiment of the invention, in response to the signal collection unit being a metal dry electrode, the specific frequency range is from 0.5 Hz to 40 HZ.

In an embodiment of the invention, the electrocardiogram detection device further comprises a communication unit. The communication unit is coupled to the microcontroller. After the filter amplifier filters out the components of the raw heartbeat signal in the specific bandwidth range, the microcontroller generates an electrocardiogram signal, and the communication unit transmits the electrocardiogram signal to an electronic device through a wireless communication protocol.

In an embodiment of the present invention, the electrocardiogram detection device further comprises a switching unit. The switching unit is coupled to the microcontroller. The switching unit outputs the trigger signal according to the signal collection unit, and the microcontroller controls the filter amplifier to filter out components of the raw heartbeat signal in different specific bandwidth ranges according to the different values of the trigger signal.

In one embodiment of the present invention, the electronic device outputs the trigger signal with a corresponding value according to the signal collection unit, the communication unit receives the trigger signal sent by the electronic device, and the microcontroller obtains the trigger signal through the communication unit.

In an embodiment of the invention, the wireless communication protocol is a Bluetooth communication protocol.

In an embodiment of the present invention, the electrocardiogram detection device further comprises a signal conversion unit. The signal conversion unit converts the raw heartbeat signal output by the filter amplifier from an analog form to a digital form and then transmits the converted raw heartbeat to the microcontroller which performs a special signal enhancement process.

In an embodiment of the present invention, the signal conversion unit converts the raw heartbeat signal output by the filter amplifier from an analog form to a digital form and then transmits the converted raw heartbeat to the microcontroller which performs a special signal enhancement process.

With regard to other additional features and advantages of the present invention, those skilled in the art can do so according to the cloud management system and the device configuration method disclosed in the implementation method of the present invention without departing from the spirit and scope of the present invention Get some changes and retouching.

### BRIEF DESCRIPTION OF THE DRAWINGS

The invention can be more fully understood by reading the subsequent detailed description and examples with references made to the accompanying drawings, wherein:

FIG. 1 is a schematic diagram showing an exemplary embodiment of an electrocardiogram detection device; and

FIG. 2 is a schematic diagram of showing an exemplary embodiment of an electrocardiogram detection device.

### DETAILED DESCRIPTION OF THE INVENTION

In order to make the above and other objects, the features and advantages of the present invention more obvious and understandable, the preferred embodiments are specifically listed below by referring to the accompanying drawings and described in detail as follows. It should be noted that this section describes the best way to implement the present invention. The purpose is to illustrate the spirit of the present invention and not to limit the scope of protection of the present invention. It should be understood that the following embodiments can be implemented by software, hardware, firmware or any combination of the above.

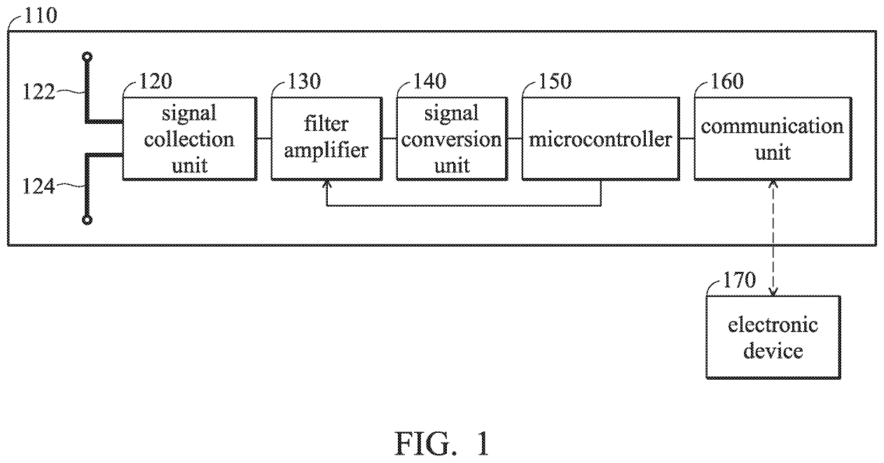

The present invention provides an electrocardiogram detection device, which adaptively adjusts a frequency range of a heartbeat signal detected by the electrocardiogram detection device according to a user's current usage situation. As shown in FIG. 1, FIG. 1 is a schematic diagram of an electrocardiogram detection device according to a first embodiment of the present invention. An electrocardiogram detection device 110 comprises a signal collection unit 120, a filter amplifier 130, a signal conversion unit 140, a microcontroller 150, and a communication unit 160.

In the embodiment of the present invention, the signal collection unit 120 may be a gel electrode or a metal dry electrode. The advantaged of the gel electrode are that it can effectively contact the body of the user and can be worn on the body for a long time for continuously recording and that the user will not feel uncomfortable. As for the metal dry electrode, it is mainly used for recording a single event. For example, in a situation where the user feels uncomfortable temporarily or a situation where the ECG is detected only for a short time, the metal electrode is used for detection.

For the gel electrode which is used in a long-term detection situation, because the contact impedance between the gel electrode and the human body is low, even if the heartbeat signal is detected at a lower frequency, the signal of the amplifier would not be saturated, thus the detection time would not be too long. In detail, the frequency range covered by the human heartbeat signal is large. For example, the signal related to myocardial infarction can be detected in the low frequency range of 0.05 Hz˜0.5 Hz. Due to the low contact impedance between the gel electrode and the human body, the contact noise can be reduced. Accordingly, even if the signals of 0.05 Hz˜0.5 Hz in the low frequency band are collected, the delay of displaying signals due to signal saturation will not occur. In other words, if the user uses the gel electrode for detection, the heartbeat signal which covers a wide frequency range can be obtained reliably within a reasonable waiting time.

In the architecture of the embodiment of the present invention, if the signal collection unit 120 is implemented by a gel electrode, a raw heartbeat signal which is in a specific bandwidth range can be obtained, wherein the specific bandwidth range is 0.05 Hz˜40 HZ.

As described above, compared with a gel electrode, a metal dry electrode is easier to clean. Therefore, for a single, short-term detection, a metal dry electrode is usually used to detection a heartbeat signal. However, since the impedance between the metal dry electrode and the human body is higher, more noise will be generated during the detection. In cases where the frequency range of the detection is large, more noise will be collected, especially in a low-frequency range of 0.05 Hz˜0.5 Hz., and, thus, the signal of the amplifier would be saturated so that the delay of displaying the signal occurs.

That is, in cases where the user uses a metal dry electrode to detect a heartbeat signal and still wants to detect the heartbeat signal in a large-frequency range, the signal may be saturated, and delay of displaying signals will occur. The user will wait for the heartbeat signal to be displayed on a display screen a long time which may be up to tens of seconds, resulting in inefficient detection. Therefore, in a situation where a metal dry electrode is used, if it is desired to shorten the detection response time, the detected frequency range needs to be adjusted appropriately.

Accordingly, the embodiment of the present invention allows the user to adjust the frequency range for detecting a heartbeat signal according to the type of the signal collection unit 120 used by the user. In detail, as shown in FIG. 1, the signal collection unit 120 comprises two heartbeat signal transmission lines 122 and 124. The heartbeat signal transmission lines 122 and 124 can be connected to the left hand and the right hand, or the left hand and the right foot, or the right hand and the left foot, respectively.

A filter amplifier 130 is connected to the signal collection unit 120. In this example, the filter amplifier 130 comprises an operational amplifier, two resistor-capacitor circuits (hereinafter referred to as a first RC circuit and a second RC circuit), and a selector, however, the present invention is not limited thereto. By adjusting the capacitance values and the resistance values of the first RC circuit and the second RC circuit, the first RC circuit and the second RC circuit can have different filtering effects. In other words, through the configurations of different resistance values and the capacitance values, the first RC circuit and the second RC circuit can enable the filter amplifier 130 to filter out the components of the signal received by the filter amplifier in different specific bandwidth ranges. The selector can enable each of the first RC circuit and the second RC circuit is turned on or off.

In the embodiment of the present invention, for example, the heartbeat signal transmission line 122 is connected to the user's left hand, and the heartbeat signal transmission line 124 is connected to the user's right hand. The signal collection unit 120 collects the raw heartbeat signal from the heartbeat signal transmission lines 122 and 124 and sends them to the filter amplifier 130.

Then, the filter amplifier 130 filters and amplifies the raw heartbeat signal. As described above, the filtered frequency range is determined or selected according to the type of signal collection unit 120 for the usage situation. The different types of signal collection units 120 correspond to different specific bandwidth ranges. When the signal collection unit 120 is implemented by a gel electrode, the specific bandwidth range is from 0.05 Hz to 40 HZ. When the signal collecting unit 120 is implemented by a metal dry electrode, the specific bandwidth range is from 0.5 Hz to 40 HZ. As described above, for example, the first RC circuit in the filter amplifier 130 can filter out the signal components in 0.05 Hz˜40 HZ, and the second RC circuit can filter out the signal components in 0.5 Hz˜40 HZ.

Since the voltage of the raw heartbeat signal is relatively small, the filter amplifier 130 further amplifies the voltage of the raw heartbeat signal to facilitate enhanced display of signals by the microcontroller 150 in the following procedure.

In the operation flow of the first embodiment of the present invention, as shown in FIG. 1, first, the user owns or holds an electronic device 170, such as a smart phone, which communicates with the electrocardiogram detection device 110 in a wireless communication protocol connection. The wireless communication protocol is a Bluetooth communication protocol in the embodiment of the present invention, however, the present invention is not limited thereto.

In the first detection situation, if the user uses a metal dry electrode to detect the heartbeat signal, through the application software installed in the electronic device 170, the user sets the current detection mode as a mode where a metal dry electrode is used (or sets the current detection mode to indicate that the signal collection unit 120 is implemented by a metal dry electrode). The electronic device 170 outputs a trigger signal with a corresponding value according to the signal collection unit 120 selected by the user. Different values of the trigger signal represent different types of signal collection units 120.

The communication unit 160 receives the trigger signal sent by the electronic device 170, and the microcontroller 150 obtains the trigger signal through the communication unit 160. Then, the microcontroller 150 generates a control signal according to the trigger signal. The control signal may be a digital signal, such as 1 or 0, to control the filter amplifier 130 to filter out the components of the raw heartbeat signal collected by the signal collection unit 120 in a specific bandwidth range. The control signal with “1” corresponds to the gel electrode, and the control signal with “0” corresponds to the metal dry electrode. For example, when the control signal sent by the microcontroller 150 is “1”, the selector will turn on the first RC circuit of the filter amplifier 130 to filter out the signal components in a specific bandwidth range of 0.05 Hz˜40 HZ. In contrast, when the control signal sent by the microcontroller 150 is “0”, the selector will turn on the second RC circuit of the filter amplifier 130 to filter out the signal components in a specific bandwidth range of 0.5 Hz˜40 HZ.

The signal conversion unit 140 is disposed between the filter amplifier 130 and the microcontroller 150. The signal conversion unit 140 converts the raw heartbeat signal output by the filter amplifier 130 from an analog form to a digital form and then transmits it to the microcontroller 150 for performing a special signal enhancement process. For the raw heartbeat signal that has been converted into the digital form and amplified, the special signal enhancement process is performed to enhance display of the P wave about atrial systole, the Q, R, and S waves about ventricular systole, and the T wave about ventricular diastole and, this, to generate an electrocardiogram signal. Then, the communication unit 160 transmits the electrocardiogram signal to the electronic device 170 through the Bluetooth communication protocol.

In the second measurement situation, if the user uses a gel electrode to detect the heartbeat signal, through the application software installed in the electronic device 170, the user sets the current detection mode as a mode where a gel electrode is used (or sets the current detection mode to indicate that the signal collection unit 120 is implemented by a gel electrode). The electronic device 170 outputs the trigger signal with a corresponding values to the electrocardiogram detection device 110, and the microcontroller 150 generates a control signal according to the trigger signal to control the filter amplifier 130 to filter out the components of the raw heartbeat signal in the corresponding specific bandwidth range of 0.05 Hz˜40 HZ. The rest of the subsequent steps are the same as the above and will not be repeated here.

In a specific embodiment, when the signal collection unit 120 is connected to the electrocardiogram detection device 110, the microcontroller 150 may determine that the signal collection unit 120 is a gel electrode or a metal dry electrode according to the electrical parameters of the signal collection unit 120.

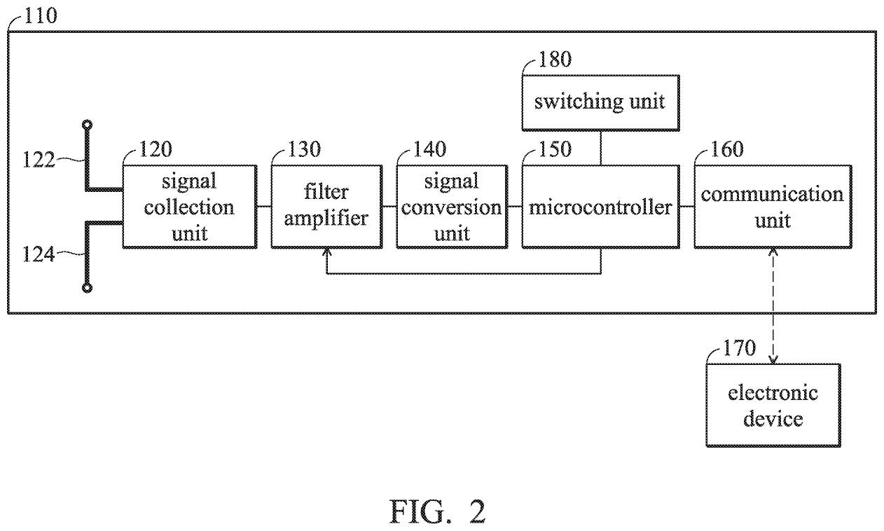

FIG. 2 is a schematic diagram of an electrocardiogram detection device according to a second embodiment of the present invention. The difference between the second embodiment and the first embodiment is that there is a switching unit 180. The switching unit 180 in the second embodiment is coupled to the microcontroller 150, and the switching unit 180 outputs a trigger signal with a corresponding value according to the signal collection unit 120 which is selected for use. The microcontroller 150 controls the filter amplifier 130 to filter out the signal components of the raw heartbeat signal in different specific bandwidth ranges according to the different values of the trigger signal. The switching unit 180 may be implemented by a physical switch (Switch). The user directly adjusts or switches the physical switch based on whether a gel electrode or a metal dry electrode is used in the current detection situation. The physical switch generates a trigger signal with a different value based on whether a gel electrode or a metal dry electrode is used. The subsequent microcontroller 150 also sends a control signal according to the trigger signal with the different value to control the filter amplifier 130 to filter out the components of the raw heartbeat signal in a specific bandwidth range.

In a specific embodiment, the switching unit 180 can be notified which one of a gel electrode and a metal dry electrode or can be triggered according to which one of a gel electrode and a metal dry electrode through different mechanisms or electrical parameters when one of the gel electrode and the metal dry electrode is connected to the electrocardiogram detection device 110, so as to automatically generate a trigger signal whose which has different values respectively in a case where gel electrode is used and a case where a metal dry electrode is used.

The present invention provides an electrocardiogram detection device, which can adaptively adjust a frequency range of a heartbeat signal detected by the electrocardiogram detection device according to the type of electrode currently used to detect the heartbeat signal, which avoids the signal saturation caused in a specific usage situations and reduces the time waiting for the display of the electrocardiogram, so that users can more efficiently know or observe whether their heartbeat signal is normal.

While the invention has been described by way of example and in terms of the preferred embodiments, it is to be understood that the invention is not limited to the disclosed embodiments. On the contrary, it is intended to cover various modifications and similar arrangements (as would be apparent to those skilled in the art). For example, the system and method described in the embodiments of the present invention may be implemented by physical embodiments of hardware, software, or a combination of hardware and software. Therefore, the scope of the appended claims should be accorded the broadest interpretation so as to encompass all such modifications and similar arrangements

What is claimed is:

1. An electrocardiogram detection device comprising:
a filter amplifier filtering and amplifies a raw heartbeat signal; and
a microcontroller coupled to the filter amplifier, wherein the microcontroller generates a control signal according to a trigger signal to control the filter amplifier to filter out components of the raw heartbeat signal in a specific bandwidth range.

2. The electrocardiogram detection device as claimed in claim 1, further comprising:
a signal collection unit, connected to the filter amplifier, collecting the raw heartbeat signal, and transmitting the raw heartbeat signal to the filter amplifier, wherein different values of the trigger signal correspond to different types of the signal collection unit.

3. The electrocardiogram detection device as claimed in claim 2, wherein in response to the signal collecting unit being a gel electrode, the specific frequency range is from 0.05 Hz to 40 HZ.

4. The electrocardiogram detection device as claimed in claim 2, when in response to the signal collection unit being a metal dry electrode, the specific frequency range is from 0.5 Hz to 40 HZ.

5. The electrocardiogram detection device as claimed in claim 2, further comprising:
a communication unit coupled to the microcontroller, wherein after the filter amplifier filters out the components of the raw heartbeat signal in the specific bandwidth range, the microcontroller generates an electrocardiogram signal, and the communication unit transmits the electrocardiogram signal to an electronic device through a wireless communication protocol.

6. The electrocardiogram detection device as claimed in claim 5, further comprising:
a switching unit coupled to the microcontroller, wherein the switching unit outputs the trigger signal according to the signal collection unit, and the microcontroller controls the filter amplifier to filter out components of the raw heartbeat signal in different specific bandwidth ranges according to the different values of the trigger signal.

7. The electrocardiogram detection device as claimed in claim 5, wherein the electronic device outputs the trigger signal with a corresponding value according to the signal collection unit, the communication unit receives the trigger signal sent by the electronic device, and the microcontroller obtains the trigger signal through the communication unit.

8. The electrocardiogram detection device as claimed in claim 5, wherein the wireless communication protocol is a Bluetooth communication protocol.

9. The electrocardiogram detection device as claimed in claim 5, further comprising:
a signal conversion unit disposed between the filter amplifier and the microcontroller, wherein the signal conversion unit converts the raw heartbeat signal output by the filter amplifier from an analog form to a digital form and then transmits the converted raw heartbeat to the microcontroller which performs a special signal enhancement process.

10. The electrocardiogram detection device as claimed in claim 9, wherein the special signal enhancement process is performed to enhance display of a P wave about atrial systole, Q, R, and S waves about ventricular systole, and a T wave about ventricular diastole.
